(19) United States
(12) Patent Application Publication
(10) Pub. No.: US 2010/0036265 A1
(43) Pub. Date: Feb. 11, 2010

(54) APPARATUS AND METHOD FOR MEASURING BLOOD PRESSURE

(75) Inventors: Youn-ho KIM, Hwaseong-si (KR); Kun-soo SHIN, Seongnam-si (KR); Seok-chan KIM, Seoul (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/469,237

(22) Filed: May 20, 2009

(30) Foreign Application Priority Data
Aug. 6, 2008 (KR) 10-2008-0076749

(51) Int. Cl. A61B 5/02

(52) U.S. Cl. 600/493

(57) ABSTRACT

A method of measuring blood pressure includes measuring a characteristic of body fat of a measured body part, detecting a sphygmus wave and a pressure of a blood vessel of the measured body part, detecting a base blood pressure of the measured body part based on a result of the detecting the sphygmus wave and the pressure of the blood vessel of the measured body part, and detecting a continuous blood pressure using a blood pressure calibration. The blood pressure calibration uses the base blood pressure and a blood pressure calculation regression equation including the characteristic of the body fat.

### CROSS-REFERENCE TO RELATED APPLICATION

This application claims priority to Korean Patent Application No. 10-2008-0076749, filed on Aug. 6, 2008, and all the benefits accruing therefrom under 35 U.S.C. §119, the contents of which in its entirety are herein incorporated by reference.

### BACKGROUND

1. Field

Embodiments disclosed herein relate to an apparatus and method for measuring blood pressure, and more particularly, to an apparatus and method for non-invasively and accurately measuring blood pressure based on a calculated characteristic of subcutaneous fat of a body part for which the blood pressure is measured.

2. Description of the Related Art

Various types of blood pressure measuring apparatuses have been developed in response to increasing interests in health. Blood pressure measuring methods include a Korotkoff sounds method, an oscillometric method and a tonometric method, for example. More particularly, in the Korotkoff sounds method, which is a typical blood pressure measuring method, a systolic pressure, at which a pulse sound is first heard, and a diastolic pressure, at which the pulse sound is barely audible, are measured in a decompression process after blood flow is blocked by sufficiently compressing a body part through which arterial blood flows.

The oscillometric method and the tonometric method are generally used for digital blood pressure measuring apparatuses. The oscillometric method, for example, detects vibration of a blood vessel which occurs in a decompression process after sufficiently compressing a body part to block arterial blood flow therethrough, similar to the Korotkoff sounds method. As a result, the oscillometric method measures systolic and diastolic blood pressure. Specifically, during the decompression process, a pressure at which an amplitude of vibration of the blood vessel is at specific levels is measured as the systolic and diastolic pressures. In the tonometric method, on the other hand, a body part is compressed such that arterial blood flow is not completely blocked, and blood pressure is thereby continually measured, based on a detected size and form of a sphygmus wave generated by compressing the body part.

Thus, the tonometric method continuously measures blood pressure, but such a method causes errors due to differences in skin thickness between a pressure sensor contacting the skin and a blood vessel, for example. More specifically, when subcutaneous fat between the pressure sensor and the blood vessel is thick, measured blood pressure is lower than an actual blood pressure. Conversely, when the subcutaneous fat between the pressure sensor and the blood vessel is thin, the measured blood pressure is higher than the actual blood pressure.

### SUMMARY

Exemplary embodiments include an apparatus and method for measuring blood pressure by considering a layer of body fat of a body part for which the blood pressure is measured by using a technology related to measuring topical body fat using light. As a result, an accuracy of the measurement of the blood pressure is substantially increased.

An exemplary embodiment includes an apparatus for measuring blood pressure, the apparatus including a pressure sensor, a light source, a light detector, a body fat measuring unit, a base blood pressure detecting unit and a continuous blood pressure detecting unit. The pressure sensor detects a sphygmus wave and a pressure of a blood vessel of a measured body part. The light source emits light toward the measured body part, and the light detector detects reflective light reflected from the measured body part. The body fat measuring unit calculates a characteristic of body fat of the measured body part based on an intensity of the reflective light. The base blood pressure detecting unit detects a base blood pressure of the measured body part based on a characteristic of the sphygmus wave and the pressure of the blood vessel detected by the pressure sensor, and the continuous blood pressure detecting unit detects a continuous blood pressure based on the base blood pressure and a blood pressure calculation regression equation including the characteristic of the body fat calculated by the body fat measuring unit.

The characteristic of the body fat may be a thickness of the body fat.

The base blood pressure detecting unit may detect the base blood pressure using an oscillometric method.

The continuous blood pressure detecting unit may detect the continuous blood pressure using a tonometric method.

The blood pressure measuring apparatus may further include a compression unit which compresses the blood vessel of the measured body part.

The measured body part may be a wrist, and the blood vessel of the measured body part may be a radial artery of the wrist.

The blood pressure measuring apparatus may further include a wrist band which receives the wrist.

The light source may include a light emitting diode (“LED”).

The blood pressure measuring apparatus may further include a plurality of light sources. Light sources of the plurality of light sources may be spaced apart from each other, and the light detector may be spaced apart from each of the light sources by gaps having different respective lengths, and the light detector may detect reflective lights corresponding to lights emitted from each of the light sources. The body fat measuring unit may calculate the characteristic of the body fat of the measured body part based on an intensity of the reflective lights.

The lights emitted from the light sources may have a same wavelength band.

When a gap between the light detector and a first light source is greater than a gap between the light detector and a second light source, an amount of light emitted from the first light source may be greater than an amount of light emitted from the second light source.

In an alternative exemplary embodiment, a method of measuring blood pressure includes measuring a characteristic of body fat of a measured body part, detecting a sphygmus wave and a pressure of a blood vessel of the measured body part, detecting a base blood pressure of the measured body part based on a result of the detecting the sphygmus wave and the pressure of the blood vessel of the measured body part, and detecting a continuous blood pressure using a blood pressure calibration. The blood pressure calibration uses the base blood pressure and a blood pressure calculation regression equation including the characteristic of the body fat.

The characteristic of the body fat may be a thickness of the body fat.

An oscillometric detection method may be used to detect the base blood pressure.

A tonometric detection method may be used to detect the continuous blood pressure.

The measured body part may be a wrist, and the blood vessel of the measured body part may be a radial artery of the wrist.

The measuring the characteristic of the body fat may include emitting light toward the measured body part, detecting reflective lights reflected from the measured body part, and calculating the characteristic of the body fat of the measured body part based on an intensity of the reflective lights reflected from the measured body part.

Light sources spaced apart from each other may emit the light toward the measured body part, a light detector spaced apart from the light sources by different gaps may detect the reflective lights corresponding to the light emitted from each of the light sources, and the characteristic of the body fat of the measured body part may be calculated based on an intensity of the reflective lights detected by the light detector

The lights emitted from the light sources may have a same wavelength band.

When a gap between the light detector and a first light source is greater than a gap between the light detector and a second light source, an amount of light emitted from the first light source may be greater than an amount of light emitted from the second light source.

### BRIEF DESCRIPTION OF THE DRAWINGS

The above and/or other aspects, features and advantages of the present invention will become more readily apparent appreciated by describing in further detail exemplary embodiments thereof with reference to the accompanying drawings, in which:

FIG. 1A is a cross-sectional view of an exemplary embodiment of a blood pressure measuring apparatus which does not compress a radial artery according to the present invention;

FIG. 1B is a cross-sectional view of an exemplary embodiment of a blood pressure measuring apparatus which compresses a radial artery according to the present invention;

FIG. 2 is a block diagram of an exemplary embodiment of a blood pressure measuring apparatus according to the present invention;

FIG. 3 is a flowchart illustrating an exemplary embodiment of a method of measuring blood pressure according to the present invention;

FIG. 4A is a flowchart illustrating an exemplary embodiment of a method of measuring a thickness of body fat of a measured body part according to the present invention;

FIG. 4B is a flowchart illustrating an exemplary embodiment of a method of an operation of measuring base blood pressure according to the present invention;

FIG. 4C is a flowchart illustrating an exemplary embodiment of a method of an operation of measuring continuous blood pressure according to the present invention;

FIG. 5 is a waveform of amplitude versus time detected by measuring blood pressure by using an oscillometric method; and

FIG. 6 is a waveform of amplitude versus time detected by measuring blood pressure using a tonometric method.

### DETAILED DESCRIPTION

The invention will now be described more fully hereinafter with reference to the accompanying drawings, in which exemplary embodiments of the invention are shown. The present invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Furthermore, relative terms, such as “lower” or “bottom” and “upper” or “top” may be used herein to describe one element's relationship to other elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on the “upper” side of the other elements. The exemplary term “lower” can, therefore, encompass both an orientation of “lower” and “upper,” depending upon the particular orientation of the figure. Similarly, if the device in one of the figures were turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning which is consistent with their meaning in the context of the relevant art and the present disclosure, and will not be interpreted in an idealized or overly formal sense unless expressly so defined herein.

Exemplary embodiments of the present invention are described herein with reference to cross section illustrations which are schematic illustrations of idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes which result, for example, from manufacturing. For example, a region illustrated or described as flat may, typically, have rough and/or nonlinear features. Moreover, sharp angles which are illustrated may be rounded. Thus, the regions illustrated in the figures are schematic in nature and their shapes are not intended to illustrate the precise shape of a region and are not intended to limit the scope of the present invention.

Hereinafter, exemplary embodiments of the present invention will be described in further detail with reference to the accompanying drawings.

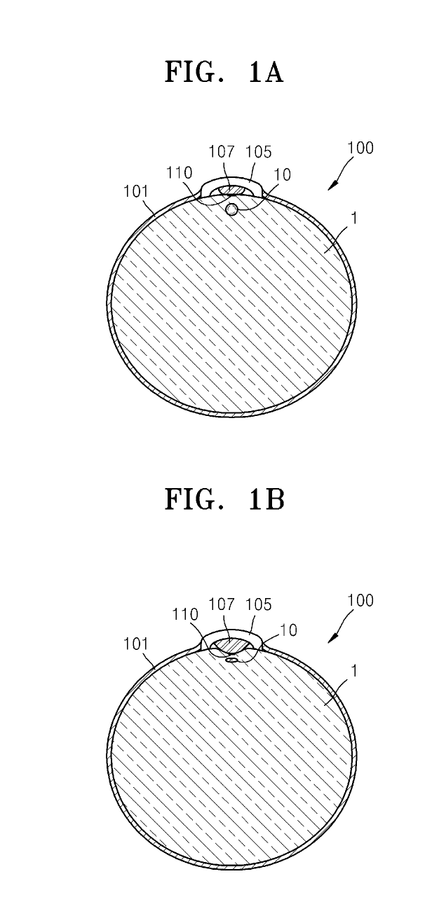

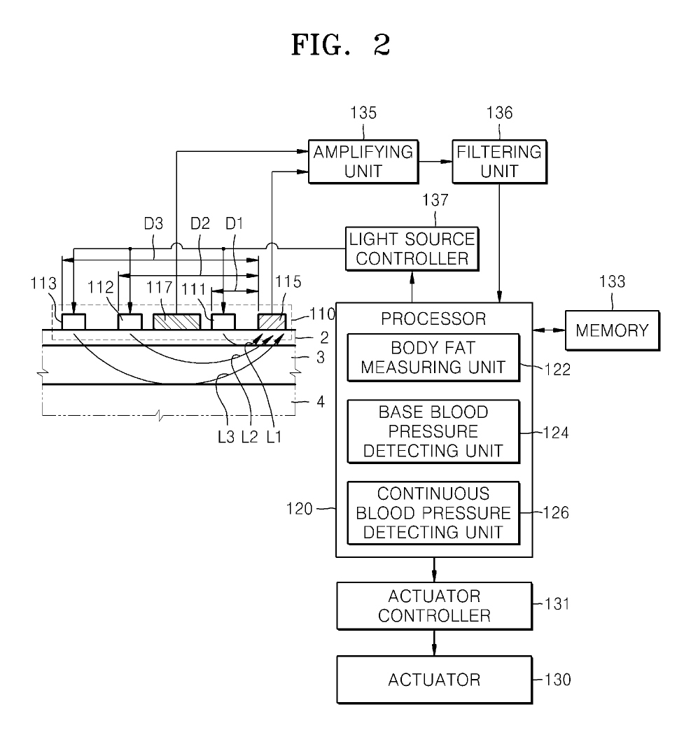

FIGS. 1A and 1B are cross-sectional views of a blood pressure measuring apparatus 100 according to the present invention. More specifically, FIG. 1A is a cross-sectional view of a blood pressure measuring apparatus 100 which does not compress a radial artery 10, while FIG. 1B is a cross-sectional view of a blood pressure measuring apparatus 100 which compresses the radial artery 10. FIG. 2 is a block diagram of the blood pressure measuring apparatus 100 according to an exemplary embodiment.

Referring to FIGS. 1A and 1B, the blood pressure measuring apparatus 100 according to an exemplary embodiment receives a wrist 1 through which the radial artery 10 is disposed, to detect a sphygmus wave and a pressure of a blood vessel, e.g., a sphygmus wave and a pressure of the radial artery 10. Thus, the blood pressure measuring apparatus 100 includes a wrist band 101, which receives and holds the wrist 1 of a subject, and a blood pressure measuring block 105 for measuring blood pressure. The blood pressure measuring block 105 includes a compression unit for compressing the radial artery 10 of the wrist 1 to be measured. The compression unit according to an exemplary embodiment includes a compression member 107, which projects toward the wrist 1 from the blood pressure measuring block 105, and an actuator 130 (FIG. 2) which drives, e.g., projects and/or contracts, the compression member 107 to move the compression member 107 toward and/or away from the wrist 1. A sensor module 110, including a plurality of sensors for measuring the blood pressure, is disposed at ends of the compression member 107 which contact the wrist 1.

Referring to FIG. 2, the sensor module 110 includes a pressure sensor 117 which detects the sphygmus wave and the pressure of the radial artery 10 (FIGS. 1A and 1B), first, second and third light sources 111, 112 and 113, respectively, which emit light toward skin 2 of the wrist 1, and a light detector 115 which detects reflective lights L1, L2 and L3 emitted from the first, second and third light sources 111, 112 and 113, respectively, and subsequently reflected back from the wrist 1 toward the light detector 115. As a result, the light detector 115 generates electrical signals corresponding to the reflective lights L1, L2 and L3.

The first, second and third light sources 111, 112 and 113, respectively, include light emitting diodes (“LEDs”) for emitting light therefrom. In an exemplary embodiment, the light emitted from first, second and third light sources 111, 112 and 113, respectively, has a same wavelength band, e.g., a same wavelength or, alternatively, a same range of wavelengths. In addition, each of the first, second and third light sources 111, 112 and 113, respectively, are spaced apart from each other, as shown in FIG. 2. Moreover, the light detector 115 is spaced apart from the first, second and third light sources 111, 112 and 113, respectively, by gaps, and the gaps each have different lengths from each other. More specifically, a first gap D1 between the light detector 115 and the first light source 111 is less than a second gap D2 between the light detector 115 and the second light source 112, while the second gap D2 is less than a third gap D3 between the light detector 115 and the third light source 113. The first, second and third gaps D1, D2 and D3, respectively, between each of the first, second and third light sources 111, 112 and 113, respectively, and the light detector 115 may vary according to a threshold value of a thickness of a portion of body fat 3 of the measured part. Further, the larger the first, second and third gaps D1, D2 and D3, respectively, are, the greater the threshold value of the thickness of the portion of body fat 3 is.

As shown in FIG. 2, the first light source 111 is disposed closest to the light detector 115 (relative to the second light source 112 and the third light source 113) and has a minimum amount of the emitted light (relative to the second light source 112 and the third light source 113). Likewise, the third light source 113 is disposed farthest from the light detector 115 (relative to the second light source 112 and the third light source 113) and has maximum amount of emitted light (relative to the second light source 112 and the third light source 113). Furthermore, the amount of light emitted from the second light source 112 is greater than that of the first light source 111, but is smaller than that of the third light source 113. In an exemplary embodiment, the amounts of the light emitted from the first, second and third light sources 111, 112 and 113, respectively, may be controlled by a light source controller 137 which adjusts a magnitude of a driving current applied to the first, second and third light sources 111, 112 and 113, respectively.

Still referring to FIG. 2, the blood pressure measuring apparatus 100 according to an exemplary embodiment further includes an amplifying unit 135 for amplifying a signal generated by the pressure sensor 117 and the light detector 115, a filtering unit 136 for removing a noise component from the amplified signal generated by the pressure sensor 117 and the light detector 115, a processor 120 for performing an operation using the filtered signal, and a memory 133 for storing data and programs necessary for the operation of the processor 120, for example. The processor 120 includes a body fat measuring unit 122 for calculating the thickness of the portion of body fat 3 of the measured part based on the intensity of the first, second and third reflective lights L1, L2 and L3, respectively, detected by the light detector 115, a base blood pressure detecting unit 124 for detecting a base blood pressure of the subject based on a characteristic of the sphygmus wave and the pressure of the radial artery 10 (FIGS. 1A and 1B) detected by the pressure sensor 117, and a continuous blood pressure detecting unit 126 for detecting a continuous blood pressure of the subject by using a blood pressure calibration which uses the base blood pressure and a blood pressure calculation regression equation which includes a value of the measured thickness of the portion of the body fat 3. The blood pressure measuring apparatus 100 further includes the actuator 130 for driving, e.g., projecting and/or contracting, the compression member 107 (FIGS. 1A and 1B), and an actuator controller 131 for controlling a driving motion of the actuator 130.

The first reflective light L1 emitted from the first light source 111 and subsequently reflected from the measured part, the second reflective light L2 emitted from the second light source 112 and subsequently reflected from the measured part, and the third reflective light L3 emitted from the third light source 113 and subsequently reflected from the measured part each have different angles incident to the light detector 115. Due to path differences of the first, second and third reflective lights L1, L2 and L3, respectively, emitted from the first, second and third light sources 111, 112 and 113, respectively, and incident on the light detector 115, the intensity of the first reflective light L1 is greater than the intensity of the second reflective light L2, and the intensity of the second reflective light L2 is smaller than the intensity of the third reflective light L3. In addition, since intensities of reflective lights reflected from different boundary surfaces, e.g., boundary surfaces between the portion of body fat 3 and muscle 4, may differ significantly. Therefore, the body fat measuring unit 122 measures the intensity of the first, second and third reflective lights L1, L2 and L3, respectively, corresponding to the first, second and third light sources 111, 112 and 113, to effectively and accurately calculate the thickness of the portion of the body fat 3 of the measured part, e.g., the wrist 1.

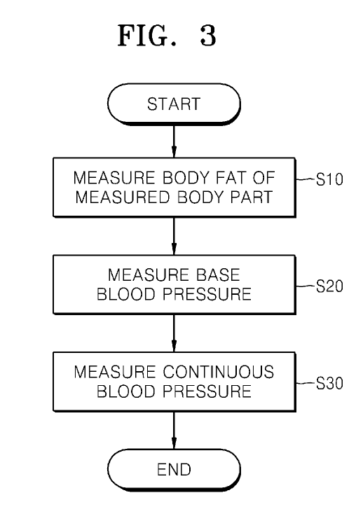

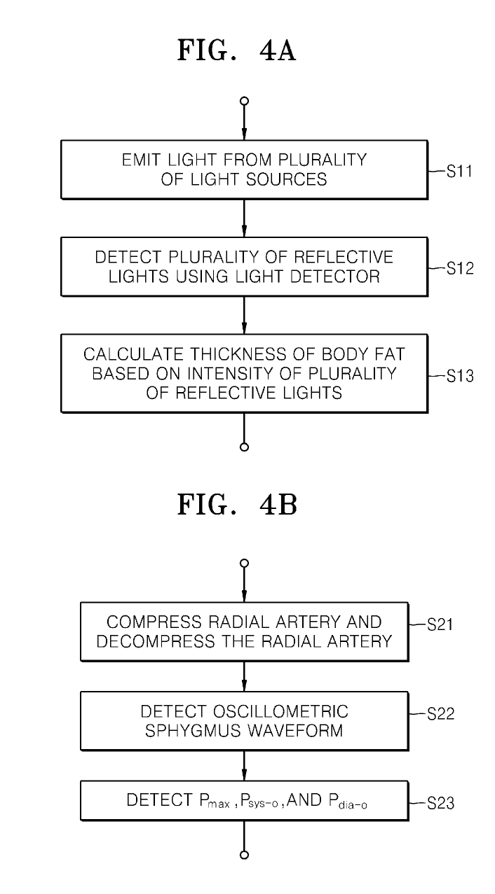

FIG. 3 is a flow chart illustrating an exemplary embodiment of a method of measuring blood pressure according to the present invention. Referring to FIG. 3, the blood pressure measuring method includes measuring the thickness of the body fat 3 of the wrist 1 in operation S10, measuring the base blood pressure in operation S20, and measuring the continuous blood pressure in operation S30, as will now be described in further detail with reference to FIGS. 2, 4A, 4B and 4C.

FIG. 4A is a flowchart illustrating an exemplary embodiment of operation S10 shown in FIG. 3. Referring to FIGS. 2 and 4A, operation S10 includes emitting light to the measured part by using the first, second and third light sources 111, 112 and 113, respectively, in operation S11, detecting the first, second and third reflective lights L1, L2 and L3, respectively, reflected from the measured part by using the light detector 115 in operation S12, and calculating the thickness of the body fat 3 of the wrist 1 based on the intensity of the detected first, second and third reflective lights L1, L2 and L3, respectively, in operation S13. The method of measuring the thickness of the body fat 3 using the first, second and third reflective lights L1, L2 and L3, respectively, and the light detector 115 were described in greater detail above with reference to FIG. 2, and thus, any repetitive detailed description thereof will hereinafter be omitted. It will be noted, however, that the inventor has performed comparative experiments by measuring thicknesses of body fat of abdomens, thighs, calves and arms of twelve (12) women by using both lights and computed tomography (“CT”) devices. The Pearson correlation between the measurement results obtained by using the lights and the CT devices is as high as about 0.9.

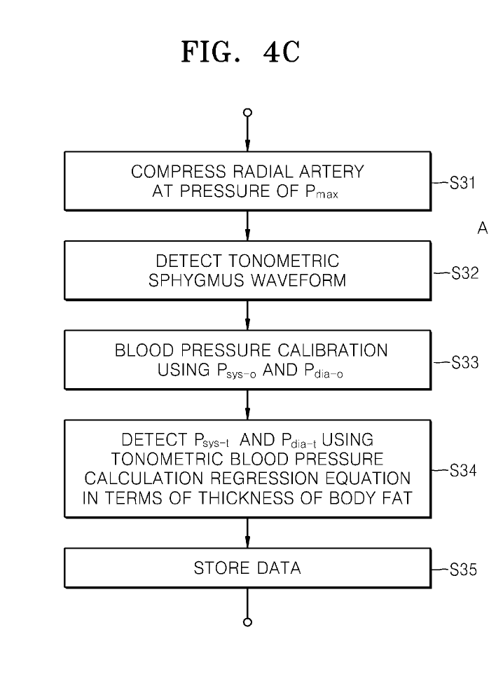

FIG. 4B is a flowchart illustrating an exemplary embodiment of operation S20 shown in FIG. 3. In an exemplary embodiment, an oscillometric detection method is used to perform operation S20. FIG. 5 is a waveform of amplitude versus time obtained by measuring blood pressure using the oscillometric detection method. Referring to FIGS. 1A, 1B, 2 and 4B, operation S20 includes compressing the radial artery 10 until it is blocked and thereafter gradually decompressing the radial artery 10 in operation S21. Moreover, an oscillometric sphygmus waveform is detected by the pressure sensor 117 during the decompression process in operation S22. The compression member 107, which is projected and/or contracted by operation of the actuator 130, compresses and/or decompresses the radial artery 10, as described in greater detail above.

The waveform shown in FIG. 5 was obtained by amplifying the oscillometric sphygmus wave detected by the pressure sensor 117, using the amplifying unit 135, and then filtering the amplified oscillometric sphygmus wave through the filtering unit 136 in operation S21. Referring to FIG. 5, an amplitude of the oscillometric sphygmus waveform over time increases, decreases and then disappears. When the oscillometric sphygmus waveform has a maximum amplitude, e.g., Aₘₐₓ, an amount of pressure detected by the pressure sensor 117 correspond to a max pressure Pₘₐₓ. When the amplitude is increased to Aₘₐₓ, or when amplitude A_{sys} is a multiple of Aₘₐₓ, e.g., A_{sys}=a(Aₘₐₓ) (where “a” is a predetermined constant less than about 1 and greater than about 0.5, such as about 0.9, for example), an amount of pressure detected by the pressure sensor 117 is P_{sys-o}, e.g., is a systolic base blood pressure measured by the oscillometric detection method. When amplitude is decreased from Aₘₐₓ to about zero (0), e.g., when amplitude A_{dia} is b times Aₘₐₓ, e.g., A_{dia}=b(Aₘₐₓ) (where b is a predetermined constant less than about 0.5 but greater than about 0, such as about 0.1, for example), an amount of pressure detected by the pressure sensor 117 is P_{dia-o}, e.g., a diastolic base blood pressure measured using the oscillometric method. Thus, the oscillometric sphygmus waveform is used to detect Pₘₐₓ, P_{sys-o}, and P_{dia-o} in operation S23.

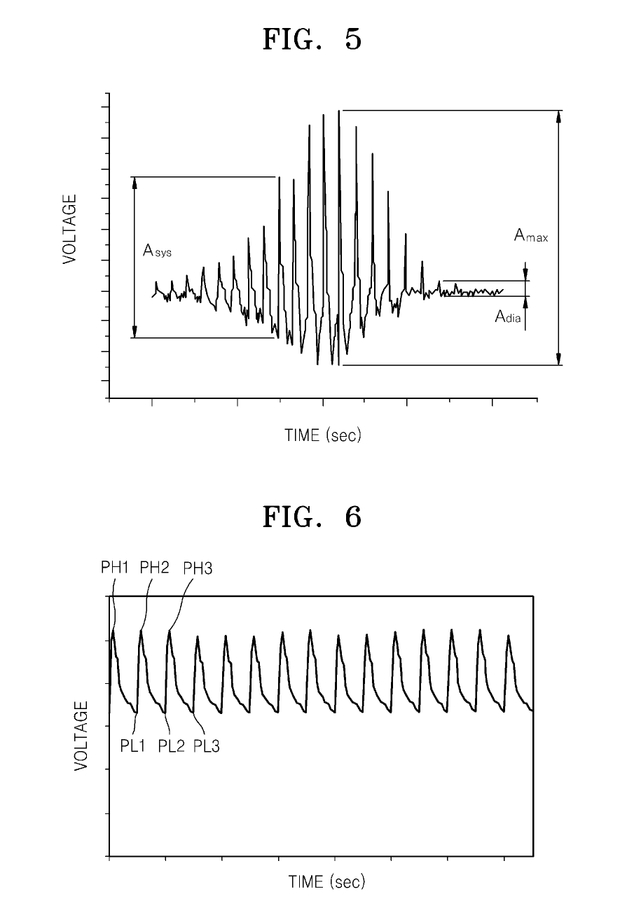

FIG. 4C is a flowchart illustrating an exemplary embodiment of operation S30 shown in FIG. 3. In an exemplary embodiment, a tonometric detection method is used to perform operation S30. FIG. 6 is a waveform of amplitude versus time detected by measuring blood pressure using the tonometric detection method. Referring to FIGS. 1A, 1B, 2 and 4C, operation S30 includes compressing the radial artery 10 to the pressure Pₘₐₓ (operation S31), and obtaining a tonometric sphygmus waveform detected by the pressure sensor 117 in operation S32. The compression member 107, which is projected and/or contracted by the operation of the actuator 130, compresses the radial artery 10.

The waveform shown in FIG. 6 was obtained by amplifying the tonometric sphygmus wave detected by the pressure sensor 117, using the amplifying unit 135, and then filtering the amplified tonometric sphygmus wave through the filtering unit 136. Referring to FIG. 6, the tonometric sphygmus waveform has a pattern including alternating high peaks (for example, PH1, PH2, PH3, etc.) and alternating low peaks (for example, PL1, PL2, PL3, etc.). In an exemplary embodiment, a systolic blood pressure P_{sys-o} is determined by measuring an amplitude at a first high peak PH1, and a diastolic blood pressure P_{dia-o} is measured by an amplitude at a first low peak PL1. The measuring of the systolic blood pressure P_{sys-o} based on the amplitude at the first high peak PH1, and the diastolic blood pressure P_{dia-o}, based on the amplitude at the first low peak PL1, is referred to as a blood pressure calibration (operation S33).

In an exemplary embodiment, an amplitude difference between the first high peak PH1 and a second high peak PH2 is used to obtain the systolic blood pressure P_{sys-o} at the second high peak PH2 by using a tonometric blood pressure calculation regression equation including a value of the measured thickness of the body fat 3. Likewise, the method is used to obtain the systolic blood pressure P_{sys-o} at a third high peak PH3 and other subsequent high peaks, as shown in FIG. 6.

Similarly, an amplitude difference between the first low peak PL1 and a second low peak PL2 is used to obtain the diastolic blood pressure P_{dia-o} at the second low peak PL2 by using the tonometric blood pressure calculation regression equation which includes the measured thickness of the body fat 3 in operation S34. Likewise, the method is used to obtain the diastolic blood pressure P_{dia-o} at a third low peak PL3 and other subsequent low peaks, as shown in FIG. 6.

In operation S34, the continuous blood pressures P_{sys-t} and P_{dia-t} are detected by alternating measurements of blood pressure, such as between the high peaks (PH1, PH2, PH3, etc.) and the low peaks (PL1, PL2, PL3, etc.), for example. The detected continuous blood pressures P_{sys-t} and P_{dia-t} are stored in the memory 133.

The blood pressure measuring apparatus 100 according to an exemplary embodiment may further include a display panel (not shown) for visually displaying blood pressure measurement results and/or the tonometric sphygmus waveform and the continuous blood pressures P_{sys-t} and P_{dia-t}.

According to exemplary embodiments of the present invention as described herein, an apparatus and method non-invasively and accurately measure blood pressure based on a calculated characteristic of subcutaneous fat of a body part for which the blood pressure is measured.

The present invention should not be construed as being limited to the exemplary embodiments set forth herein. Rather, these exemplary embodiments are provided so that this disclosure will be thorough and complete and will fully convey the concept of the present invention to those skilled in the art

While the present invention has been particularly shown and described with reference to exemplary embodiments thereof, it will be understood that the exemplary embodiments described herein are descriptive only and are not for purposes of limitation.

Thus, although exemplary embodiments have been shown and described herein, it will be understood by those of ordinary skill in the art that various changes in form and detail may be made therein without departing from the spirit or scope of the present invention as defined by the following claims.

What is claimed is:

1. An apparatus for measuring blood pressure, the apparatus comprising:
a pressure sensor which detects a sphygmus wave and a pressure of a blood vessel of a measured body part;
a light source which emits light toward the measured body part;
a light detector which detects reflective light reflected from the measured body part;
a body fat measuring unit which calculates a characteristic of body fat of the measured body part based on an intensity of the reflective light;
a base blood pressure detecting unit which detects a base blood pressure of the measured body part based on a characteristic of the sphygmus wave and the pressure of the blood vessel detected by the pressure sensor; and
a continuous blood pressure detecting unit which detects a continuous blood pressure based on the base blood pressure and a blood pressure calculation regression equation including the characteristic of the body fat calculated by the body fat measuring unit.

2. The apparatus of claim 1, wherein the characteristic of the body fat is a thickness of the body fat.

3. The apparatus of claim 1, wherein the base blood pressure detecting unit detects the base blood pressure using an oscillometric method.

4. The apparatus of claim 1, wherein the continuous blood pressure detecting unit detects the continuous blood pressure using a tonometric method.

5. The apparatus of claim 1, further comprising a compression unit which compresses the blood vessel of the measured body part.

6. The apparatus of claim 1, wherein
the measured body part is a wrist, and
the blood vessel of the measured body part is a radial artery of the wrist.

7. The apparatus of claim 6, further comprising a wrist band which receives the wrist.

8. The apparatus of claim 1, wherein the light source comprises a light emitting diode.

9. The apparatus of claim 1, further comprising a plurality of light sources, wherein
light sources of the plurality of light sources are spaced apart from each other,
the light detector is spaced apart from each of the light sources by gaps having different respective lengths,
the light detector detects reflective lights corresponding to lights emitted from each of the light sources, and
the body fat measuring unit calculates the characteristic of the body fat of the measured body part based on an intensity of the reflective lights.

10. The apparatus of claim 1, wherein the lights emitted from the light sources have a same wavelength band.

11. The apparatus of claim 9, wherein, when a gap between the light detector and a first light source is greater than a gap between the light detector and a second light source, an amount of light emitted from the first light source is greater than an amount of light emitted from the second light source.

12. A method of measuring blood pressure, the method comprising:
measuring a characteristic of body fat of a measured body part;
detecting a sphygmus wave and a pressure of a blood vessel of the measured body part;
detecting a base blood pressure of the measured body part based on a result of the detecting the sphygmus wave and the pressure of the blood vessel of the measured body part; and
detecting a continuous blood pressure using a blood pressure calibration, wherein
the blood pressure calibration comprises using the base blood pressure and a blood pressure calculation regression equation including the characteristic of the body fat.

13. The method of claim 12, wherein the characteristic of the body fat is a thickness of the body fat.

14. The method of claim 12, wherein the detecting the base blood pressure comprises an oscillometric detection method.

15. The method of claim 12, wherein the detecting the continuous blood pressure comprises a tonometric detection method.

16. The method of claim 12, wherein
the measured body part is a wrist, and
the blood vessel of the measured body part is a radial artery of the wrist.

17. The method of claim 12, wherein the measuring the characteristic of the body fat comprises:
emitting light toward the measured body part;
detecting reflective lights reflected from the measured body part; and
calculating the characteristic of the body fat of the measured body part based on an intensity of the reflective lights reflected from the measured body part.

18. The method of claim 17, wherein
light sources spaced apart from each other emit the light toward the measured body part,
a light detector spaced apart from the light sources by different gaps detects the reflective lights corresponding to the light emitted from each of the light sources, and
the characteristic of the body fat of the measured body part is calculated based on an intensity of the reflective lights detected by the light detector.

19. The method of claim 18, wherein the lights emitted from the light sources have a same wavelength band.

20. The method of claim 18, wherein, when a gap between the light detector and a first light source is greater than a gap between the light detector and a second light source, an amount of light emitted from the first light source is greater than an amount of light emitted from the second light source.
